(19) United States
(12) Patent Application Publication
(10) Pub. No.: US 2022/0340963 A1
(43) Pub. Date: Oct. 27, 2022

(54) METHODS FOR ASSESSING GRAFT SUITABILITY FOR TRANSPLANTATION

(71) Applicants: Paula E. NORTH, Wauwatosa, WI (US); Michael E. MITCHELL, Wauwatosa, WI (US); Aoy T. MITCHELL, Wauwatosa, WI (US); Paul G. DAFT, Wauwatosa, WI (US); Donna K. MAHNKE, Wauwatosa, WI (US); Natera, Inc., San Carlos, CA (US)

(72) Inventors: Paula E. NORTH, Wauwatosa, WI (US); Michael E. MITCHELL, Wauwatosa, WI (US); Aoy T. MITCHELL, Wauwatosa, WI (US); Paul G. DAFT, Wauwatosa, WI (US); Donna K. MAHNKE, Wauwatosa, WI (US)

(73) Assignee: Natera, Inc., San Carlos, CA (US)

(21) Appl. No.: 17/761,311

(22) PCT Filed: Sep. 21, 2020
(86) PCT No.: PCT/US2020/051847

§ 371 Date: Mar. 17, 2022

Related U.S. Application Data
(60) Provisional application No. 62/903,049, filed on Sep. 20, 2019.

(51) Int. Cl. C12Q 1/6851

(52) U.S. Cl. CPC C12Q 1/6851; C12Q 1/6848

(57) ABSTRACT

This invention relates to methods and compositions for assessing the suitability of a graft for transplantation by measuring total and/or specific cell-free nucleic acids (such as cf-DNA) and/or cell lysis. Specifically, the method comprising obtaining an amount of total short fragment cf-DNA and/or graft-specific short fragment cf-DNA released from a potential graft (e.g., ex vivo), e.g., prior to contacting of the potential graft with blood cells of a potential recipient, and/or subsequent to contacting of the potential graft or cells thereof with blood cells from a potential recipient, and assessing the amount(s) to determine the suitability of the potential graft

### CROSS-REFERENCE TO RELATED APPLICATIONS

This application claims priority to and the benefit of U.S. Provisional Patent Application No. 62/903,049, filed on Sep. 20, 2019, and entitled “METHODS FOR ASSESSING GRAFT SUITABILITY FOR TRANSPLANTATION”, the entire contents of the which is incorporated herein by reference.

### FIELD

Provided herein are compositions and methods for assessing graft suitability for transplantation. In particular, provided herein are methods for assessing graft suitability for transplantation by measuring total and/or graft-specific cell-free nucleic acids, such as cell-free DNA.

### BACKGROUND

Transplantation has emerged as a viable therapeutic option for the treatment of various diseases and is the only mode of therapy for most end-stage organ failures. However, transplantation rejection is an impediment to long-term transplantation success. Acute transplantation rejection can occur within days to weeks after transplantation and may lead to chronic rejection and gradual loss of function of the transplanted organ. In spite of widespread use of potent immunosuppressive drugs and improved organ preservation techniques, rejection remains a major complication post-transplantation. Accordingly, methods for identifying grafts that are suitable for transplantation are needed.

### SUMMARY

Provided herein are compositions and methods for assessing graft suitability for transplantation. In some aspects, provided herein is a method of assessing the suitability of a potential graft for transplantation. That assessment may be done by identifying the absolute or steady-state level of cell-free DNA. Total cell free DNA of a graft ex vivo in a perfusion container may include contents indicative of lysis and/or apoptosis of cells from the graft and from blood from the donor. As a graft starts to deteriorate, apoptosis of cells may increase and total cell free DNA levels may also increase. The levels can be evaluated by comparison to a threshold of cellular or blood components, for example a threshold level of cfDNA. A suitable graft for transplant would preferably have low or steady-state levels of total cell free DNA.

The method comprises obtaining an amount of total short fragment cf-DNA and/or graft-specific short-fragment cf-DNA released from a graft and assessing the amount(s) to determine the suitability of the graft for transplantation. The amount may be obtained prior to contacting the graft or cells thereof with blood cells from a potential recipient and/or subsequent to contacting the graft or cells thereof with blood cells from a potential recipient. In some embodiments, obtaining an amount comprises performing a method to quantify the amount of total short fragment cf-DNA and/or graft-specific short fragment cf-DNA released by the graft. In some aspects, the methods further comprise obtaining an amount of cell-lysis, such as by performing fragment analysis.

In some embodiments, the graft is monitored over time. In some aspects, the methods described herein may involve obtaining an amount of total short fragment cf-DNA and/or graft-specific short-fragment cf-DNA and/or determining the amount of cell lysis at one or more additional time points. In such embodiments, the method may further comprise comparing the amount(s) of the total short fragment cf-DNA and/or graft-specific cf-DNA and/or amount of cell lysis with threshold value(s) or amount(s) obtained from the additional time point(s).

In some aspects, the amount(s) of the total short fragment cf-DNA, graft-specific cf-DNA, and/or cell lysis is measured using a quantitative amplification method. In other aspects, the amount(s) is measured using next-generation sequencing.

In some aspects, the amount(s) of the total short fragment cf-DNA and/or graft-specific cf-DNA and/or amount of cell lysis are obtained in a sample of media in which the potential graft or cells therefrom are contained or are in contact with. For example, the media may be a storage, perfusion, or preservation media in which the graft is contained or in contact with.

In some embodiments, the graft is an organ or organs. For example, the graft may be a heart, a lung, or a heart and a lung. The graft may be from a donor of the same species as the potential recipient.

In any of the embodiments described herein, the method may further comprise providing the values for the amount(s) of total cell-free DNA and/or graft-specific cf-DNA and/or cell lysis in a report. The report may be used by clinicians to assess the suitability of the graft for transplantation. In some embodiments, based on information in the report, the graft is transplanted into a subject if the graft is identified as suitable for transplantation.

### BRIEF DESCRIPTION OF THE DRAWINGS

FIG. 1 shows reference gene qPCR measuring the percentage of detectable DNA after spiking a known quantity of sheared gDNA into STEEN solution™ containing heparin or no heparin.

FIG. 2 shows reference gene qPCR measuring the percentage of detectable DNA after spiking 20 ng of sheared gDNA into STEEN Solution™ supplemented with heparin at 0, 0.984, 1.125, and 1.5 IU/ml.

FIG. 3 shows reference gene qPCR measuring the percentage of detectable DNA after spiking 500 ng/ml of sheared gDNA into STEEN Solution™ containing 0, 1, 5, or 100 IU/ml of heparin.

FIG. 4 shows reference gene qPCR measuring the percent recovery of a short fragment DNA control from STEEN Solution™ containing 0, 1, 5, or 100 IU/ml of heparin.

FIG. 5 shows reference gene qPCR measuring the DNA concentration of purified and concentrated spiked STEEN solution™ samples vs. unpurified 0.1X TE controls at concentrations of 500 and 1000 ng/ml.

FIG. 6 shows reference gene qPCR measuring the DNA concentration of purified and concentrated spiked STEEN solution™ samples vs unpurified 0.1X TE controls at concentrations of 10 and 40 ng/ml.

FIG. 7 shows reference gene qPCR measuring DNA concentration in STEEN Solution™ and plasma after 0, 2, 4, and 6-hour incubations at 37° C.

FIG. 8 shows reference gene qPCR measuring DNA concentrations in STEEN solution™ after incubation at 22° C. (RT) or 37° C. for 0, 1, 3, 12, and 24 hours.

FIG. 9 shows reference gene qPCR measuring DNA concentrations in plasma after incubation at 22° C (RT) or 37° C for 0, 1, 3, 12, and 24 hours.

### DETAILED DESCRIPTION

Embodiments of the present disclosure provide compositions and methods for assessing graft suitability for transplantation. In particular, the present disclosure provides compositions and methods for assessing cell-free nucleic acids in a graft and assessing suitability of the graft for transplantation based upon these measurements.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. Many embodiments herein are described using open “comprising” language. Such embodiments encompass multiple closed “consisting of ”and/or “consisting essentially of” embodiments, which may alternatively be claimed or described using such language. The present disclosure also contemplates other embodiments “comprising,” “consisting of ”and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

“Subject” and “patient” as used herein interchangeably refers to any vertebrate, including, but not limited to, a mammal and a human. In some embodiments, the subject is a human or a non-human. “Mammal” as used herein refers to any member of the class Mammalia, including, without limitation, humans and nonhuman primates such as chimpanzees and other apes and monkey species; farm animals such as cattle, sheep, pigs, goats, llamas, camels, and horses; domestic mammals such as dogs and cats; laboratory animals including rodents such as mice, rats, rabbits, guinea pigs, and the like. The subject may be any age or sex.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Methods for Assessing Graft Suitability

Total cell free DNA of a graft ex vivo in a perfusion container generally will represent lysis and/or apoptosis of cells from the graft and any cells from blood from the donor. Without being bound by theory, it is thought that as a graft starts to deteriorate, apoptosis of cells increases, and total cell free DNA levels also increases. A suitable graft for transplant generally has low or steady-state levels of total cell free DNA.

The present disclosure provides methods for assessing graft suitability for transplantation. The methods comprise obtaining an amount of cell-free nucleic acids (such as cell-free DNA) released from a graft and assessing the amount to determine the suitability of the potential graft for transplantation. In some embodiments, the cell-free nucleic acids are short-fragment (e.g. less than or equal to 170 bps) cell-free DNA. In particular embodiments, the methods comprise obtaining an amount of short fragment (e.g. fragments less than or equal to 170 bps) cell-free DNA released from a graft. The amount of short fragment cell-free DNA is then used to assess suitability of the potential graft for transplantation.

In some embodiments, the methods provided herein or otherwise known in the art can be used multiple times to obtain total and/or specific cell-free nucleic acid (such as DNA) values over time. Also included herein are reports that include one or more of these values. Such reports provide valuable information to a clinician. In some embodiments, the clinician then assesses the condition (or suitability of a graft) and/or makes treatment decisions accordingly for a subject.

As used herein, “graft” refers to a biological material comprising cells or tissue, such as at least a portion of an organ, that may be transplanted or implanted in or into a subject. In some embodiments, the graft is explanted material comprising cells or tissue, such as at least a portion of an organ that is being maintained outside the body (ex vivo), such as to preserve or rehabilitate, the graft. Organs and tissues include, but are not limited to, heart, kidney, liver, lung, pancreas, intestine, thymus, bone, tendon, cornea, skin, heart valve, nerve, and blood vessel (e.g., vein).

In some embodiments of any one of the methods provided herein, the graft is a whole organ or more than one organ. Examples of organs that can be transplanted or implanted include, but are not limited to, the heart, kidney(s), kidney, liver, lung(s), pancreas, intestine, etc. In some embodiments, the graft is more than one organ. For example, the graft may be a combination of a heart and lung. In some embodiments, the graft is one organ. For example, the graft may be a lung. In other embodiments of any one of the methods provided herein, the graft a portion of an organ. For example, the graft may be a valve.

Grafts may be of the same species as the potential recipient of the graft, or may be of a different species from the potential recipient of the graft. In some embodiments, the graft is from a different species (i.e., is a xenograft) than the recipient. For example, the graft may be from a pig or cow and the recipient may be a human. Any one of the types of grafts provided herein may be a xenograft. In some embodiments of any one of the methods provided herein, the graft is a pig or cow valve. In other embodiments of any one of the methods provided herein, the graft is from the same species as the potential recipient of the graft. For example, the graft may be from a human and the recipient may be a different human.

In other embodiments of any one of the methods provided herein the graft is decellularized graft, such as a decellularized xenograft. In some embodiments of any one of the methods provided herein the graft is an autograft. Any one of the methods or compositions provided herein may be used for assessing any one of the grafts described herein. Any one of the methods provided herein can be used to evaluate suitability for future engraftment. For example, the methods provided herein can be used to evaluate suitability of a graft isolated from one subject for suitability for engraftment into another subject.

In some embodiments, the methods described herein comprise obtaining a value of cell-free DNA from a sample obtained from or in contact with the graft. For example, the methods comprise obtaining a value of short fragment cell-free DNA from a sample obtained from or in contact with the graft. As used herein, the sample can be a biological sample. Examples of such biological samples include whole blood, plasma, serum, etc. In some embodiments of any one of the methods provided herein, the sample comprises media in which the graft is placed or with which it has contact. In some embodiments, the media comprises blood or a blood substitute, preservation solution, or any other solution in which a graft is placed or with which it has contact, such as in in vitro contexts. In some embodiments, the graft, such as an organ or organs, is contained in a perfusion system.

In some embodiments of any one of the methods provided herein, the one or more samples and/or one or more other samples are obtained within minutes, such as no more than 15, 20, 25, 30, 35, 40, 45, 50, or 55 minutes, of obtaining the graft (e.g., storing the graft, perfusing the graft, etc.).

In some embodiments of any one of the methods provided herein, the one or more samples and/or one or more other samples are obtained within hours, such as no more than 1, 2, 3, 4, 5, 6, 7, 8, 9, 10, 12, 18 or more hours, of obtaining the graft (e.g., storing the graft, perfusing the graft, etc.)

In some embodiments of any one of the methods provided herein, more than one sample is obtained. For example, an initial sample may be obtained, and one or more additional samples may be obtained at subsequent time points. In some embodiments, the one or more other subsequent time points are at hourly intervals. For example, in some embodiments an initial sample is obtained within an hour of obtaining the graft and one or more other samples are obtained within 15, 20, 25, 30, 35, 40, 45, 50, or 55 minute intervals or 1, 2, 3, 4, 5, 6, 7, 8, 9, 10, 12, 18 or more hourly intervals, such as until a threshold value or baseline is reached. In some embodiments of any one of the methods provided herein, the one or more other subsequent time points are at daily intervals. In some embodiments of any one of the methods provided herein, the one or more other subsequent time points are at one-week intervals. In some embodiments of any one of the methods provided herein, the one or more other subsequent time points are at two-week intervals. In some embodiments of any one of the methods provided herein, the one or more other subsequent time points are at monthly intervals.

The sample may be subjected to various methodologies prior to obtaining the level of cell-free nucleic acids in the sample. For example, the sample may be subjected to one or more centrifugation steps. In some embodiments, the sample is centrifuged once. In some embodiments, the sample is centrifuged more than once. For example, the sample may be centrifuged once, two times, three times, four times, five times, or more than five times prior to obtaining the value of cell-free DNA in the sample. The centrifugation steps may be performed at any suitable speed for any suitable duration to achieve the desired result. For example, the sample may be centrifuged at 1000×g, 1100×g, 1200×g, 1300×g, or 1400×g. In some embodiments, the sample is centrifuged at the desired speed within 2 hours of sample collection. In some embodiments, the sample is centrifuged once or multiple times at a first sample collection site and shipped to a second site for quantification of cell-free nucleic acids in the sample. Additional centrifugation steps may occur at the second site, such as after thawing the sample. For example, the sample may be centrifuged 2-3 times at 1100×g, 1200×g, 1300×g, or 1400×g within 2 hours of collection, frozen, and shipped to a second site for quantification of cell-free nucleic acids in the sample.

In some embodiments of any one of the methods provided herein, the graft (e.g., cells, tissue, organ) is maintained in graft storage media. Graft storage media can be intracellular (e.g., perfused) or extracellular, and may depend on the graft to be preserved. Approaches to preserving most grafts include simple static cold storage (SCS) and dynamic preservation. Examples of dynamic preservation include hypothermic machine perfusion (HMP), normothermic machine perfusion, and oxygen persufflation.

Typically, in combination with hypothermia, graft storage media can prevent clotting in harvests with blood present, reduce stress and deterioration associated with ex vivo handling, and decrease the risk of microbial growth. Therefore, in some embodiments, the graft storage media comprises osmotic active agents, electrolytes, hydrogen ion buffers, colloid(s), metabolic inhibitors, metabolites, and antioxidants. Examples of osmotic active agents, which may prevent cell swelling, include lactobionate, raffinose, citrate, and gluconate. Electrolytes, which can exert an osmotic effect, include sodium, potassium, calcium, and magnesium ions. Examples of hydrogen ion buffers include phosphate, histidine, and N-(2-hydroxyethyl)-piperazine-N′-2-ethanesulfonic acid (HEPES) buffer. Examples of colloids, which may be used during the initial vascular flush out and perfusion, include albumin and HES. Examples of metabolic inhibitors, which may suppress degradation of cell constituents, include allopurinol, antiproteases, and chlorpromazine.

Examples of metabolites, which can help restore metabolism during the reperfusion phase, include adenosine, glutathione, and phosphate. Examples of antioxidants, which can inhibit oxygen free-radical injury, include steroids, vitamin E, deferoxamine, and tryptophan.

Graft storage media are commercially available, and examples include BELZER UW® cold storage solution (VIASPAN™ or the University of Wisconsin (UW) solution), CELSIOR®, CUSTODIOL®, and IGL-1®.

In particular embodiments, the graft is a lung or lungs. The compositions and methods provided herein may be used to determine suitability of donor lungs for transplantation. In particular embodiments, the graft is one or more ex-vivo lung perfusion (EVLP) lungs. In some embodiments, the methods are applied to EVLP lungs that have been removed from one subject to evaluate suitability for engraftment into another subject. Accordingly, provided herein is a method for monitoring cellular injury in ex-vivo lung perfusion (EVLP) lungs during perfusion to assess suitability of the lung for transplantation.

In some embodiments, the methods described herein are used to evaluate the potential contribution of cf-DNA released from leukocytes or other cell types emanating from the perfused lungs, as well as potential interference with cf-DNA analysis by molecular additives to the EVLP system, such as heparin or dextran. In some embodiments, these preparatory determinations are used to provide accurate reporting of total cf-DNA in EVLP perfused lungs, and donor fraction in lung transplant patients.

During EVLP, the perfusion circuit of the lung mimics in vivo conditions. In some embodiments, the ventilated ex vivo lungs are perfused with STEEN™ solution without red blood cells. The parameters of gaseous exchange, pulmonary vascular resistance, compliance, and other key variables under normothermic conditions are monitored. STEEN™ solution is a buffered extracellular solution that includes human serum albumin (HSA) to provide optimal osmotic pressure, facilitated by Dextran 40, a mild scavenger to coat and protect the endothelium from excessive leucocyte interaction. STEEN™ solution is designed to facilitate prolonged evaluation of lung transplantation options and to and promote health of isolated lungs ex vivo. EVLP using STEEN™ solution thus has the potential to be able to increase the likelihood that previously rejected , but ex vivo rehabilitated lungs could be used to increase the availability of potential organs for lung transplantation. Accordingly, in some embodiments the sample is STEEN™ solution that has been in contact with the graft.

In some embodiments, the methods for assessing suitability of a graft for transplantation described herein comprise obtaining an amount of cell-free nucleic acids released from a graft and assessing the amount of cell-free nucleic acid to determine the suitability of the potential graft for transplantation. In some aspects, the methods include obtaining a value for the amount of total cell-free nucleic acids (such as DNA) and/or a value for the amount of specific cell-free nucleic acids (such as DNA).

As used herein, a “value” is any indicator that conveys information about an “amount.” The indicator can be an absolute or relative value for the amount. As used herein, “amount” refers to the quantity of nucleic acids (such as DNA). Further, the value can be the amount, frequency, ratio, percentage, etc.

In some instances the values can be compared to a “threshold value.” As used herein, a “threshold value” refers to any predetermined level or range of levels that is indicative of a state, the presence or absence of a condition or the presence or absence of a risk. The threshold value can take a variety of forms. It can be single cut-off value, such as a median or mean. It can be established based upon comparative groups, such as where the risk in one defined group is double the risk in another defined group. As another example, a threshold value is a baseline value, such as without the presence of a state, condition or risk or after a course of treatment or other remedial action. Such a baseline can be indicative of a normal or other state not correlated with the risk or condition or state that is being tested for.

As used herein, “specific cell-free nucleic acids” refers to a subset of cell-free nucleic acids (such as DNA) that is within the total cell-free nucleic acids (such as DNA). In some embodiments, the specific cell-free nucleic acids (such as DNA) are cell-free nucleic acids (such as DNA) that are graft-specific (GS). GS cf-DNA refers to DNA that presumably is shed from the graft or cells thereof, the sequence of which matches (in whole or in part) the genotype of the subject from which the graft is obtained. As used herein, GS cf-DNA may refer to certain sequence(s) in the GS cf-DNA population, where the sequence is distinguishable from the recipient or potential recipient cf-DNA (e.g., having a different sequence at a particular nucleotide location(s)), or it may refer to the entire GS cf-DNA population). In some embodiments, the GS cf-DNA is short fragment (e.g. less than or equal to 170 bp) GS cf-DNA.

The values for the amount(s) of nucleic acids (such as DNA) can be “obtained” by any one of the methods provided herein or other methods known in the art, and any obtaining step(s) can include any one of the methods incorporated herein by reference or otherwise provided herein. “Obtaining” as used herein refers to any method by which the respective information or materials are acquired. Thus, the respective information (e.g. the amount of cell-free DNA) can be acquired by experimental methods. Respective materials can be created, designed, etc. with various experimental or laboratory methods, in some embodiments. The respective information or materials can also be acquired by being given or provided with the information, such as in a report, or materials. For example, the amount of cell-free DNA can be obtained by being provided with a report containing the value of the amount of cell-free DNA in a sample. In some embodiments, materials are given or provided through commercial means (i.e. by purchasing the materials from a third party service laboratory).

As provided herein, the suitability can be determined using one or more values for the amount of total cell-free nucleic acids (such as DNA) and/or one or more values for the amount of specific cell-free nucleic acids (such as DNA).

Ideally, most of the cell free DNA to be analyzed will come from the organ, and the blood will have washed away. However, intact leukocytes from the donor can still be present in the organ. Also, lysis of cells can lower the quality of the perfusate for total cell free DNA analysis. Thus, in some embodiments of any one of the methods provided herein, contaminating intact cells are removed from samples, such as perfusate samples, by one or more (e.g., one, two or three or more) centrifugation steps. In some embodiments of any one of the methods provided herein, a baseline is established for meaningful cf-DNA analysis after effective washout of contaminating leukocytes.

The suitability can also be determined using one or more values for the amount of total cell-free nucleic acids (such as DNA) and/or one or more values from fragment analysis. Cell lysis, such as from mechanical stress and degenerative changes after sample collection, as opposed to apoptosis, releases long genomic fragments in biological samples, such as blood samples. On the other hand, the majority of cf-DNA from blood drawn from normal healthy individuals appears to be the result of normal cellular apoptosis and turnover. Apoptosis results in the release of relatively short DNA fragments, significantly shorter than those released by cellular lysis. Samples in which significant cell lysis has occurred (e.g. samples that contain higher proportions of long fragments) may not be suitable for accurate analysis of amounts of cell-free DNA. Accordingly, in some embodiments, fragment analysis is performed to evaluate potential contaminating factors, such as excess long fragment DNA, that may negatively impact the ability to accurately quantify cf-DNA in the graft.

In some embodiments, fragment analysis is performed by assessing short and/or long nucleic acid fragments. As used herein, a “long fragment” refers to a fragment that is greater than 170 bps (e.g., between 171 and 300 bps in length), while a “short fragment” is a fragment that is less than or equal to 170 bps. For example, a short fragment may be between 75 and 170 bps in length. Such methods generally are performed with primers targeting a long fragment and/or a short fragment.

The fragment can be an Alu fragment. An Alu element is a short stretch of DNA originally characterized by the action of the Arthrobacter luteus (Alu) restriction endonuclease. Alu repeats are the most abundant sequences in the human genome, with a copy number of about 1.4 million per genome. Alu sequences are short interspersed nucleotide elements (SINEs), typically 300 nucleotides, which account for more than 10% of the genome. Provided herein are methods that in some embodiments include measuring the potential contaminating contribution of cell lysis of a cf-DNA sample by analyzing long Alu fragments and/or short Alu fragments.

In some embodiments, a high proportion of long fragment DNA in the sample may negatively impact accurate cf-DNA measurements. Accordingly, in some embodiments, the methods comprise obtaining an amount or a value of short-fragment cell-free nucleic acids in the sample. For example, long DNA fragments are removed from the sample and the remaining short-fragment cell-free DNA are measured. Alternatively, short DNA fragments are isolated from the sample and short-fragment cell-free nucleic acids are subsequently measured. Any suitable materials and methods for removing long DNA fragments or isolating short DNA fragments in the sample may be used. Suitable materials and methods include, for example, size-based purification methods including gel electrophoresis, particles (e.g. paramagnetic particles), beads, resins, and the like. For example, suitable methods include silica-based methods, (e.g. silica beads, paramagnetic silica particles, lysine-functionalized silica particles, PEG-modified silica particles, carboxyl-functionalized silica particles, lysine-functionalized silica particles, silica matrixes, amine-modified silica matrixes, metal ion-modified silica matrixes, etc.) to preferentially isolate short DNA and/or remove long-DNA from the sample. In some embodiments, bead-based size-selective purification is performed to remove long DNA fragments and/or isolate short DNA fragments. For example, long DNA fragments are removed by bead isolation, as described in Example 12. In accordance with such embodiments, the total cell-free nucleic acids and/or specific cell-free nucleic acids is quantified using short-fragment DNA in the sample. This value is used to assess suitability of the graft for transplantation.

In some embodiments of any one of the methods provided, the method further includes assessing the suitability (e.g., health, state, or condition) of a graft for transplantation based on the value(s) of cell-free nucleic acids (e.g. cell-free DNA) obtained. In some embodiments, assessing the suitability comprises providing a determination of the suitability of the graft for transplantation. The determination may be binary (e.g. suitable or not suitable). For example, the suitability of a graft for transplantation may be assessed based upon the value of short-fragment cell-free DNA obtained. In some embodiments, any one of the methods provided herein comprises correlating an increase in one or more values (e.g., for an amount of total and/or specific cell-free nucleic acids (such as DNA)) with unsuitability or declining suitability or a decrease in one or more values (e.g., for an amount of total and/or specific cell-free nucleic acids (such as DNA)) with suitability or increasing suitability. For example, in some embodiments, an increased value of short-fragment cell-free DNA (e.g. total and/or specific short fragment cell-free DNA) in a sample is correlated with unsuitability or declining suitability of a graft for transplantation. As another example, a decrease in short-fragment cell-free DNA (e.g. total and/or specific short-fragment cell-free DNA) in a sample is correlated with suitability or increasing suitability of a graft for transplantation.

In some embodiments, the amount of long DNA fragments, short DNA fragments, and the amount of total and/or specific short fragment cell-free DNA is obtained and used to assess the suitability of a graft for transplantation. For example, the ratio of long: short DNA fragments (or short: long DNA fragments) is obtained and the amount of total and/or specific short fragment cell-free DNA is obtained, and the combination of these values is used to assess suitability of a graft for transplantation into a subject. For example, an increased ratio of long:short fragment DNA and an increased amount of total and/or specific short fragment cell-free DNA indicates unsuitability or diminished suitability of a graft for transplantation.

In some embodiments of any one of the methods provided herein, correlating comprises comparing a level (e.g., concentration, ratio or percentage) to a threshold value or value from another point in time to determine suitability, or increasing or decreasing suitability. Thus, changes in the levels can be monitored over time. Any one of the methods provided herein can include one or more steps of comparing the values for an amount of nucleic acids (such as DNA) to a threshold value or a value from a different point in time to assess the suitability of the graph. In some embodiments of any one of the methods provided herein, the method may further include additional test(s) for assessing suitability of the graph. The additional test(s) may employ any one of the methods provided herein or methods known in the art.

It has been found that particularly useful to a clinician is a report that contains the value(s) provided herein. In some embodiments, the reports include one or more threshold values. Reports may be provided in oral, written (or hard copy) or electronic form, such as in a form that can be visualized or displayed. In some embodiments, the “raw” results for each assay as provided herein are provided in a report, and from this report, further steps can be taken to analyze the amount(s) nucleic acids (such as DNA). In other embodiments, the report provides multiple values for the amounts of nucleic acids (such as DNA). From the amounts, in some embodiments, a clinician may assess the suitability of a graft for transplantation or the need to monitor the graft over time or treatment or some other remedial action. In some embodiments, the methods comprise transplanting the graph or starting, ending, or modifying treatment based on the results.

In some embodiments, the amounts are in or entered into a database. In one aspect, a database with such values is provided. From the amount(s), a clinician may assess the need for a treatment or monitoring. Accordingly, in any one of the methods provided herein, the method can include assessing the amount(s) at more than one point in time. Such assessing can be performed with any one of the methods or compositions provided herein.

In any one of the methods provided herein, the method can include assessing the amount of nucleic acids (such as DNA) at another point in time or times. Such assessing can be performed with any one of the methods provided herein.

Methods for determining total cell-free nucleic acids (such as DNA) as well as specific cell-free nucleic acids (such as DNA) are provided herein or are otherwise known in the art. For example, the methods of PCT Application No. PCT/US2016/030313, herein incorporated by reference in its entirety, may be used for determining a value for the amount of specific cell-free nucleic acids (such as DNA) in a sample as provided herein. Thus, any one of the methods provided herein may include the steps of any one of the methods described in PCT Application No. PCT/US2016/030313 Likewise, the methods of measuring cell-free DNA of U.S. Publication No. US-2015-0086477-A1 are also incorporated herein by reference and such methods can be included as part of any one of the methods provided herein for determining a value for the amount of cell-free nucleic acids (such as DNA). As a further example, amplification with PCR, such as real-time PCR or digital PCR, may be used to determine a value for the amount of total cell-free nucleic acids (such as DNA) and/or specific cell-free nucleic acids (such as DNA). In some embodiments, the quantification is obtained for each target relative to a standard, such as an internal standard, that is spiked into a sample(s). For example, in some embodiments of any one of the methods provided herein, the total cell-free nucleic acids (such as DNA) is determined with Taqman Real-time PCR using RNase P as a target. Other methods are provided elsewhere herein or would be apparent to those of ordinary skill in the art. Any one of the methods provided herein, can include any one of the methods of determining a value provided herein.

As mentioned above, in some embodiments, any one of the methods provided herein may include steps of a quantitative assay that makes use of mismatch amplification (e.g., MOMA) in order to determine a value for an amount of specific cell-free nucleic acids (such as DNA).

In some embodiments of any one of such mismatch methods, the method further comprises selecting informative results of the amplification-based quantification assays, such as PCR quantification assays. In some embodiments of any one of such mismatch methods, the selected informative results are averaged, such as a median average. In some embodiments of any one of such mismatch methods, the results can be further analyzed with Robust Statistics. In some embodiments of any one of such mismatch methods, the results can be further analyzed with a Standard Deviation, such as a Robust Standard Deviation, and/or Coefficient of Variation, such as a Robust Coefficient of Variation, or % Coefficient of Variation, such as a % Robust Coefficient of Variation.

In some embodiments of any one of such mismatch methods, the informative results of the amplification-based quantification assays, such as PCR quantification assays are selected based on the genotype of the non-specific nucleic acids and/or specific nucleic acids.

In some embodiments of any one of such mismatch methods, the method further comprises obtaining the genotype of the non-specific nucleic acids and/or specific nucleic acids.

In some embodiments of any one of such mismatch methods, there is at least one primer pair, at least two primer pairs, at least three primer pairs, at least four primer pairs or more per SNV target. In some embodiments of any one of such mismatch methods, the plurality of SNV targets is at least 45, 48, 50, 55, 60, 65, 70, 75, 80, 85 or 90 or more. In some embodiments of any one of such mismatch methods, the plurality of SNV targets is at least 90, 95 or more targets. In some embodiments of any one of such mismatch methods, the plurality of SNV targets is less than 90, 95 or more targets. In some embodiments of any one of such mismatch methods, the plurality of SNV targets is less than 105 or 100 targets.

In some embodiments of any one of such mismatch methods, the mismatched primer(s) is/are the forward primer(s). In some embodiments of any one of such mismatch methods, the reverse primers for the primer pairs for each SNV target is the same.

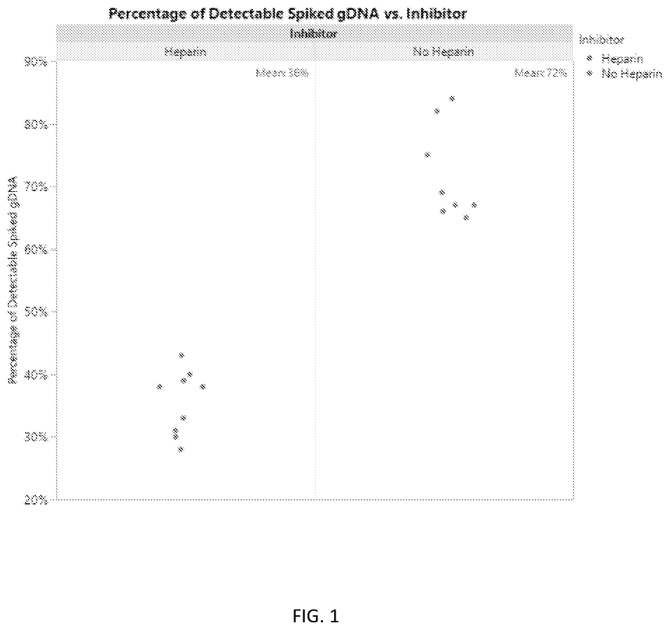

Primers for use in such assays may be obtained, and any one of the methods provided herein can include a step of obtaining one or more primer pairs for performing the quantitative assays. Generally, the primers possess unique properties that facilitate their use in quantifying amounts of nucleic acids. For example, a forward primer of a primer pair can be mismatched at a 3′ nucleotide (e.g., penultimate 3′ nucleotide). In some embodiments of any one of the methods provided, this mismatch is at a 3′ nucleotide but adjacent to the SNV position. In some embodiments of any one of the methods provided, the mismatch positioning of the primer relative to a SNV position is as shown in FIG. 1. Generally, such a forward primer even with the 3′ mismatch to produce an amplification product (in conjunction with a suitable reverse primer) in an amplification reaction, thus allowing for the amplification and resulting detection of a nucleic acid with the respective SNV. If the particular SNV is not present, and there is a double mismatch with respect to the other allele of the SNV target, an amplification product will generally not be produced. Preferably, in some embodiments of any one of the methods provided herein, for each SNV target a primer pair is obtained whereby specific amplification of each allele can occur without amplification of the other allele(s).

“Specific amplification” refers to the amplification of a specific target without substantial amplification of another nucleic acid or without amplification of another nucleic acid sequence above background or noise. In some embodiments, specific amplification results only in the amplification of the specific allele. As used herein, “single nucleotide variant” refers to a nucleic acid sequence within which there is sequence variability at a single nucleotide. In some embodiments, the SNV is a biallelic SNV, meaning that there is one major allele and one minor allele for the SNV. In some embodiments, the SNV may have more than two alleles, such as within a population. Generally, a “minor allele” refers to an allele that is less frequent in a set of nucleic acids, for a locus, while a “major allele” refers to the more frequent allele in a set of nucleic acids. The methods provided herein can quantify nucleic acids of major and minor alleles within a mixture of nucleic acids even when present at low levels, in some embodiments.

The nucleic acid sequence within which there is sequence identity variability, such as a SNV, is generally referred to as a “target”. As used herein, a “SNV target” refers to a nucleic acid sequence within which there is sequence variability at a single nucleotide. The SNV target has more than one allele, and in preferred embodiments, the SNV target is biallelic. In some embodiments of any one of the methods provided herein, the SNV target is a SNP target. In some of these embodiments, the SNP target is biallelic. In some embodiments of any one of the methods provided, the amount of nucleic acids is determined by attempting amplification-based quantitative assays, such as quantitative PCR assays, with primers for a plurality of SNV targets. A “plurality of SNV targets” refers to more than one SNV target where for each target there are at least two alleles. Preferably, in some embodiments, each SNV target is expected to be biallelic and a primer pair specific to each allele of the SNV target is used to specifically amplify nucleic acids of each allele, where amplification occurs if the nucleic acid of the specific allele is present in the sample.

In some embodiments of any one of the methods provided herein, for each SNV target that is biallelic, there are two primer pairs, each specific to one of the two alleles and thus have a single mismatch with respect to the allele it is to amplify and a double mismatch with respect to the allele it is not to amplify (again if nucleic acids of these alleles are present). In some embodiments of any one of the methods provided herein, the mismatch primer is the forward primer. In some embodiments of any one of the methods provided herein, the reverse primer of the two primer pairs for each SNV target is the same.

These concepts can be used in the design of primer pairs for any one of the methods provided herein. It should be appreciated that the forward and reverse primers are designed to bind opposite strands (e.g., a sense strand and an antisense strand) in order to amplify a fragment of a specific locus of the template. The forward and reverse primers of a primer pair may be designed to amplify a nucleic acid fragment of any suitable size to detect the presence of, for example, an allele of a SNV target according to the disclosure. Any one of the methods provided herein can include one or more steps for obtaining one or more primer pairs as described herein.

Generally, “informative results” as provided herein are the results that can be used to quantify the level of nucleic acids in a sample. In some embodiments of any one of the methods provided, the amount of specific- and/or non-specific nucleic acids represents an average across informative results for the nucleic acids, respectively. In some embodiments of any one of the methods provided herein, this average is given as an absolute amount or as a percentage. Preferably, in some embodiments of any one of the methods provided herein, this average is the median.

The amount, such as ratio or percentage, of specific nucleic acids may be determined with the quantities of the major and minor alleles as well as genotype, as needed. In some embodiments of any one of the methods provided herein, the alleles can be determined based on prior genotyping (e.g., of the recipient or potential recipient and/or the subject from which a graft is obtained, respectively). Methods for genotyping are well known in the art. Such methods include sequencing, such as next generation, hybridization, microarray, other separation technologies or PCR assays. Any one of the methods provided herein can include steps of obtaining such genotypes.

It should be appreciated that the primer pairs described herein may be used in a multiplex assays, such as multiplex PCR assays. Accordingly, in some embodiments, the primer pairs are designed to be compatible with other primer pairs in a PCR reaction. For example, the primer pairs may be designed to be compatible with at least 2, at least 5, at least 10, at least 20, at least 30, at least 40, etc. other primer pairs in a PCR reaction. As used herein, primer pairs in a PCR reaction are “compatible” if they are capable of amplifying their target in the same PCR reaction. In some embodiments, primer pairs are compatible if the primer pairs are inhibited from amplifying their target nucleic acid (such as DNA) by no more than 1%, no more than 2%, no more than 5%, no more than 10%, no more than 15%, no more than 20%, no more than 25%, no more than 30%, no more than 35%, no more than 40%, no more than 45%, no more than 50%, or no more than 60% when multiplexed in the same PCR reaction. Primer pairs may not be compatible for a number of reasons including, but not limited to, the formation of primer dimers and binding to off-target sites on a template that may interfere with another primer pair.

Accordingly, the primer pairs of the disclosure may be designed to prevent the formation of dimers with other primer pairs or limit the number of off-target binding sites. Exemplary methods for designing primers for use in a multiplex assays are known in the art and are otherwise described herein.

In some embodiments of any one of the methods provided herein, the mismatch amplification-based quantitative assay is any quantitative assay whereby nucleic acids are amplified and the amounts of the nucleic acids can be determined. Such assays include those whereby nucleic acids are amplified with the MOMA primers as described herein and quantified. Such assays include simple amplification and detection, hybridization techniques, separation technologies, such as electrophoresis, next generation sequencing and the like.

In some embodiments of any one of the methods provided herein, the quantitative assays include quantitative PCR assays. Quantitative PCR include real-time PCR, digital PCR, TaqMan, etc. In some embodiments of any one of the methods provided herein the PCR is “Real-time PCR”. Such PCR refers to a PCR reaction where the reaction kinetics can be monitored in the liquid phase while the amplification process is still proceeding. In contrast to conventional PCR, real-time PCR offers the ability to simultaneously detect or quantify in an amplification reaction in real time. Based on the increase of the fluorescence intensity from a specific dye, the concentration of the target can be determined even before the amplification reaches its plateau.

In any one of the methods provided herein the PCR may be digital PCR. Digital PCR involves partitioning of diluted amplification products into a plurality of discrete test sites such that most of the discrete test sites comprise either zero or one amplification product. The amplification products are then analyzed to provide a representation of the frequency of the selected genomic regions of interest in a sample. Analysis of one amplification product per discrete test site results in a binary “yes-or-no” result for each discrete test site, allowing the selected genomic regions of interest to be quantified and the relative frequency of the selected genomic regions of interest in relation to one another be determined. In certain aspects, in addition to or as an alternative, multiple analyses may be performed using amplification products corresponding to genomic regions from predetermined regions. Results from the analysis of two or more predetermined regions can be used to quantify and determine the relative frequency of the number of amplification products. Using two or more predetermined regions to determine the frequency in a sample reduces a possibility of bias through, e.g., variations in amplification efficiency, which may not be readily apparent through a single detection assay. Methods for quantifying DNA using digital PCR are known in the art and have been previously described, for example in U.S. Patent Publication No. US20140242582, which is herein incorporated by reference in its entirety.

Any one of the methods provided herein can comprise extracting nucleic acids, such as cell-free DNA. Such extraction can be done using any method known in the art or as otherwise provided herein (see, e.g., Current Protocols in Molecular Biology, latest edition, or the QlAamp circulating nucleic acid kit or other appropriate commercially available kits). An exemplary method for isolating cell-free DNA from blood is described. Blood containing an anti-coagulant such as EDTA or DTA is collected. The plasma, which contains cf-DNA, is separated from cells present in the blood (e.g., by centrifugation or filtering). An optional secondary separation may be performed to remove any remaining cells from the plasma (e.g., a second centrifugation or filtering step). The cf-DNA can then be extracted using any method known in the art, e.g., using a commercial kit such as those produced by Qiagen. Other exemplary methods for extracting cf-DNA are also known in the art (see, e.g., Cell-Free Plasma DNA as a Predictor of Outcome in Severe Sepsis and Septic Shock. Clin. Chem. 2008, v. 54, p. 1000-1007; Prediction of MYCN Amplification in Neuroblastoma Using Serum DNA and Real-Time Quantitative Polymerase Chain Reaction. JCO 2005, v. 23, p. 5205-5210; Circulating Nucleic Acids in Blood of Healthy Male and Female Donors. Clin. Chem. 2005, v. 51,p. 1317-1319; Use of Magnetic Beads for Plasma Cell-free DNA Extraction: Toward Automation of Plasma DNA Analysis for Molecular Diagnostics. Clin. Chem. 2003, v. 49, p. 1953 -1955; Chiu RWK, Poon LLM, Lau TK, Leung TN, Wong EMC, Lo YMD. Effects of blood-processing protocols on fetal and total DNA quantification in maternal plasma. Clin Chem 2001;47:1607 -1613; and Swinkels et al. Effects of Blood-Processing Protocols on Cell-free DNA Quantification in Plasma. Clinical Chemistry, 2003, vol. 49, no. 3, 525-526). In some embodiments, the long DNA fragments are removed from the sample and the remaining short fragment cell-free DNA are extracted.

In some embodiments of any one of the methods provided herein, a pre-amplification step is performed. An exemplary method of such a pre-amplification is as follows, and such a method can be included in any one of the methods provided herein. Approximately 15 ng of cell-free plasma DNA is amplified in a PCR using Q5 DNA polymerase with approximately 13 targets where pooled primers were at 4 uM total. Samples undergo approximately 25 cycles. Reactions are in 25 ul total. After amplification, samples can be cleaned up using several approaches including AMPURE bead cleanup, bead purification, or simply ExoSAP-IT™, or Zymo.

In some embodiments, provided herein is a kit. The kit may comprise components necessary, useful, or sufficient to perform one or more aspects of the methods described herein. For example, the kit may comprise any one or more of sample collection tubes, centrifuge tubes, sample storage tubes, packaging materials necessary for shipping the sample, materials necessary for isolating short-fragment DNA and/or removing long fragment DNA from the sample (e.g. beads, particles, resins, buffers, etc.) materials for quantifying cell-free DNA in the sample (e.g. primers, probes, buffers, etc.), devices and software for measuring cell-free DNA, and instructions for using the kit.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in some embodiments may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different from illustrated, which may include performing some acts simultaneously, even though shown as sequential acts in illustrative embodiments.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The following description provides examples of the methods provided herein.

### 3. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

### Evaluation of PCR Inhibition by Dextran 40

The Toronto EVLP method utilized involves gradual re-warming of the lungs to normal core body temperature in conjunction with gradual increase in vascular flow, targeting a perfusion flow of 40% donor-predicted cardiac output (CO). It utilizes protective lung ventilation and an acellular perfusate with increased colloid osmotic pressure achieved through inclusion of human serum albumin and Dextran 40. This widely applied methodology is FDA approved under a humanitarian device exemption (HOE).

During EVLP, the perfusion circuit of the lung mimics in vivo conditions. The ventilated ex vivo lungs are perfused with STEEN™ solution without red blood cells. The parameters of gaseous exchange, pulmonary vascular resistance, compliance, and other key variables under normothermic conditions are monitored. Six hours of EVLP, even more, is clinically considered standard when using an acellular STEEN perfusate. STEEN™ solution is a buffered extracellular solution that includes human serum albumin (HSA) to provide optimal osmotic pressure, facilitated by Dextran 40, a mild scavenger to coat and protect the endothelium from excessive leucocyte interaction. STEEN™ solution is designed to facilitate prolonged evaluation of lung transplantation options and to and promote health of isolated lungs ex vivo. EVLP using STEEN™ solution thus has the potential to be able to increase the likelihood that previously rejected , but ex vivo rehabilitated lungs could be used to increase the availability of potential organs for lung transplantation.

Procedure and Results: Dextran 40 (MW 40,000) was solubilized in H2O at weight to weight concentrations of 50%, 25%, 12.5%, 6.25%, 3.13%, 1.56% and 0.78%. Each of these Dextran concentrations was spiked with 20 ng of sheared gDNA, and measured directly for total DNA levels using reference gene qPCR. Results are shown in Table 1.

TABLE 1
Reference gene qPCR measuring detectable gDNA
in H2O supplemented with 0.78-50% Dextran 40.

| Percent | Spiked | Mean of spiked |
|---|---|---|
| Dextran 40 | DNA (ng) | gDNA Detected (ng) |
| 0 | 20 | 18.5 |
| 0.78% | 20 | 0 |
| 1.56% | 20 | 0 |
| 3.13% | 20 | 0 |
| 6.25% | 20 | 0 |
| 12.5% | 20 | 0 |
| 25% | 20 | 0 |
| 50% | 20 | 0 |

Conclusions: Dextran 40 is completely inhibitory of the reference gene qPCR assay at concentrations as low as 0.78%. This data suggests that the Dextran 40 contained in STEEN solution™ (at unknown concentration), if not removed from the DNA fraction by extraction, might have a significant an inhibitory effect on measurement of cfDNA in EVLP circuit samples. Example 2 below was designed to evaluate that possibility.

### Example 2

### Quantification of Human DNA in STEEN Solution™ as Measured by Feference Gene qPCR Method

Procedure and Results: One ml volumes of STEEN solution™ supplemented with 0 or 1.5 IU/ml heparin were spiked with gDNA sheared to approximate cfDNA size (average 150 bp). Spiked samples were measured directly without extraction for total DNA levels using reference gene qPCR. Results are shown in FIG. 1.

Conclusions and Subsequent Actions: Spiked gDNA sheared to approximate cfDNA size was detected by qPCR in STEEN Solution™ without extraction. However, when heparin was present at concentrations seen in an EVLP circuit (1.5 IU/ml), detection of DNA was significantly diminished.

In order to report the cfDNA concentration in a clinical EVLP circuit as a general indicator of tissue injury and acceptability for transplantation, the cfDNA concentration in the EVLP perfusate solution should be quantified rapidly to be useful for clinical decision making. To meet this need for speed, it is important to develop a rapid DNA assay that does not include traditional extraction of the DNA measurement independent of DNA extraction. This data suggests that DNA can be measured by qPCR from STEEN Solution™ without need to undergo a time-consuming extraction step.

### Example 3

### Quantification of Sheared Human gDNA in STEEN Solution at Heparin Concentrations Equivalent to Those in an EVLP Circuit

To better understand the findings of Example 2, we evaluated the ability to detect spiked sheared gDNA in STEEN containing heparin concentrations equivalent to pre- and post-replenishment of the EVLP circuit.

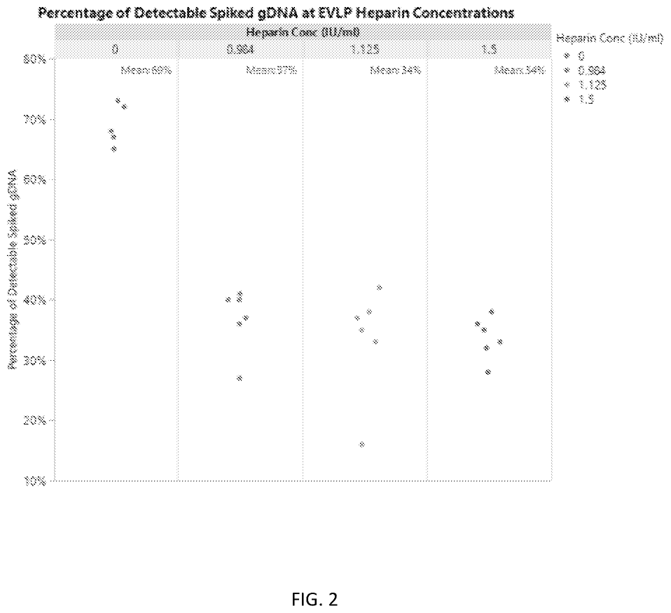

Procedure and Results: One ml volumes of STEEN solution™ supplemented with heparin at 0, 0.984, 1.125, or 1.5 IU/ml were spiked with sheared gDNA. Spiked samples were measured directly using a reference gene qPCR method without extraction. Results are shown in FIG. 2.

Conclusions and Subsequent Actions: The inhibition of qPCR by heparin is observed at all concentrations observed in a 3-hour EVLP circuit. Heparin is a highly sulfonated glycosaminoglycan with the ability to bind a wide range of biomolecules including: DNA binding proteins such as initiation factors, elongation factors, restriction endonucleases, DNA ligase, DNA, and RNA polymerases. Because heparin is negatively charged like the phosphodiester bridges of DNA, it is possible that heparin inhibits the qPCR reaction by binding directly to DNA polymerase. If this is true, a bead-based DNA purification step to directly bind DNA and allow removal of background heparin would increase the percentage of detectable spiked gDNA (Example 4).

### Example 4

### Quantification of Bead-Purified Human DNA in STEEN Solution Supplemented with Heparin.

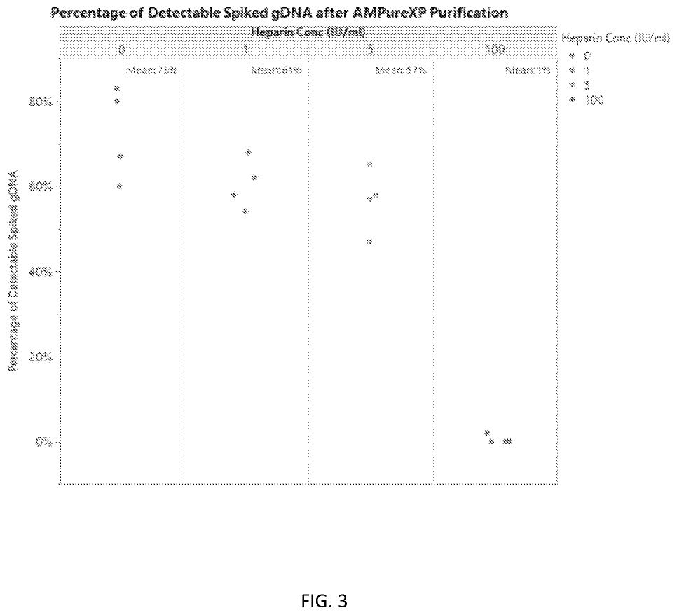

Procedure and Results: STEEN Solution supplemented with 0, 1, 5, or 100 IU/ml heparin was spiked with 500 ng/ml of sheared gDNA. The DNA from these samples was then purified. Purification can be done using various methods, including bead purification. In this example, purification was done using AMPureXP beads and eluted in nuclease free water. The purified DNA was measured directly for total DNA levels using a reference gene qPCR method. Results are shown in FIG. 3.

Conclusions and Subsequent Actions: The binding of DNA by bead-based purification appears to allow for separation from background heparin and provides a method to rapidly measure total cfDNA content in patient samples without the need for DNA extraction. Spiked gDNA is detected at heparin concentrations up to 5 IU/ml, which is >3-fold higher than concentrations in an EVLP circuit. However, without the extraction process concentrating DNA from a sample, a perfusate concentration of 100 ng/ml (100pg/μl ) only contains amplifiable DNA equivalent to 10-15 cells. It is possible that perfusates from an

EVLP circuit could be at even lower concentrations, which could result in non-ideal PCR conditions. Understanding DNA levels from EVLP perfusate samples from an EVLP circuit that have been prepared is useful in determining if bead-based purification is a viable option.

### Example 5

### Quantification of Short DNA Fragment Control in STEEN Solution After Bead Purification

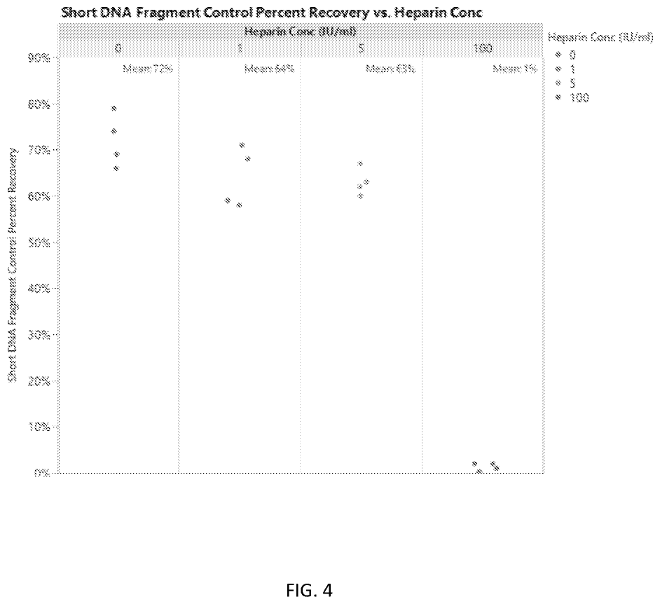

Procedure and Results 5: STEEN solution supplemented with 0, 1, 5, or 100 IU/ml heparin was spiked with 25,000 copies of short DNA fragment control, which closely resembles cfDNA. The DNA from these samples was then purified using AMPureXP beads and eluted in nuclease free water. The percentage of detectable short DNA fragment control was measured directly after purification using a reference gene qPCR method. Results are shown in FIG. 4.

Conclusions and Subsequent Actions: The data in the figure above demonstrates that purification of a heparin containing solution with AMPureXP beads is capable of detecting short DNA fragments in STEEN™ solution.

### Example 6

### Quantification of Bead-Purified Human DNA at Low Concentrations

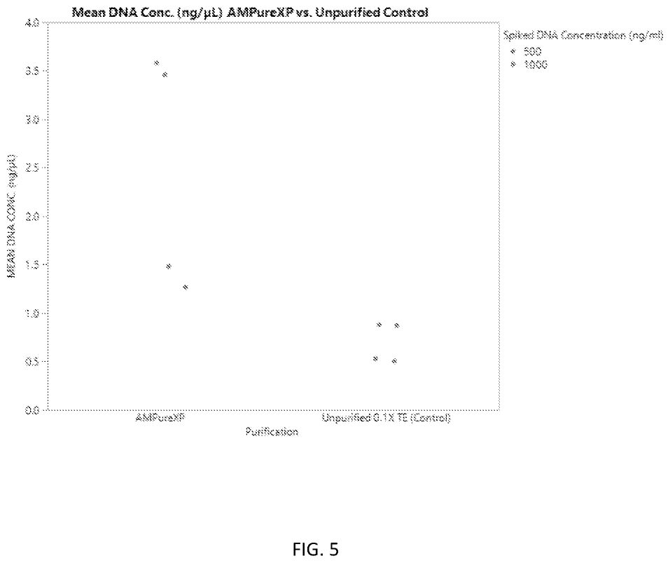

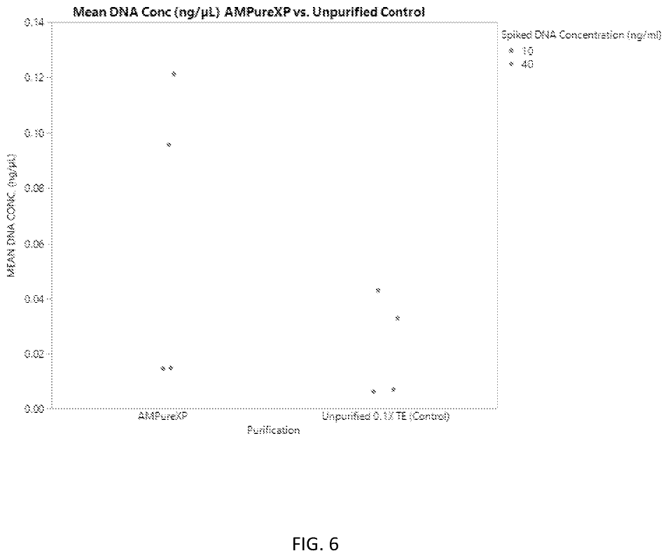

Procedure and Results: 100 μl aliquots of STEEN solution were spiked with sheared gDNA at concentrations of 0, 10, 40, 500, and 1000 ng/ml. In addition, a 0.1X TE control was spiked at equivalent concentrations. The DNA from the spiked STEEN samples was then purified using AMPureXP beads and eluted in 30 μl of nuclease free water. The purified DNA from STEEN solution and the unpurified DNA from 0.1X TE was then measured directly to determine the eluted DNA concentration using a reference gene qPCR method. Results are shown in FIG. 5 and FIG. 6.

Conclusions and Subsequent Actions: By eluting AMPureXP beads with bound DNA in 30 μl compared to the original 100 μl pre-purification volume, we are able to concentrate samples. This allows for efficient PCR quantification by reference gene at lower

DNA concentrations.

### Example 7

### Evaluation of White Blood Cell Stability in STEEN Solution Compared to Plasma

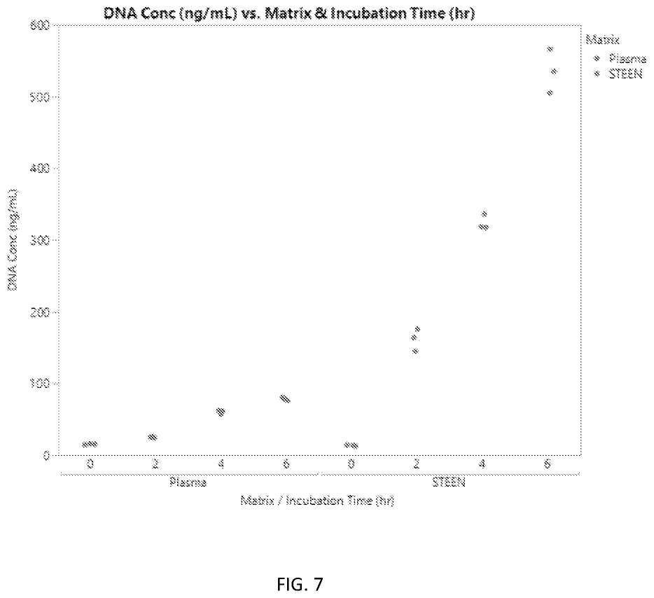

Procedure and Results: Eight ml volumes of STEEN Solution and plasma were spiked with freshly isolated buffy containing WBCs. Samples were then incubated at 37° C. on a tube revolver for 0, 2, 4, and 6 hours. After each indicated timepoint samples underwent two 1100 x g low speed centrifugations and a 15000×g high speed centrifugation. All samples were then frozen at −80° C. and extracted using an automated DNA extraction workflow. Eluates from the extractions were then measured using a reference gene qPCR method. Results are shown in FIG. 7.

Conclusions and Subsequent Actions: WBCs undergo significant amounts of lysis in STEEN when compared to plasma in as little as 2 hours. This trend continues at 4 and 6 hours. DNA levels are higher from lysing cells in STEEN Solution compared to plasma, which could be a byproduct of DNA stability within the two matrices. If DNA is degraded in plasma and not STEEN, it could appear that more lysis is occurring in STEEN. To better understand the findings of example 6, we measured the stability of DNA in STEEN solution in example 8.

### Example 8

### Evaluation of DNA Stability in STEEN Solution and Plasma

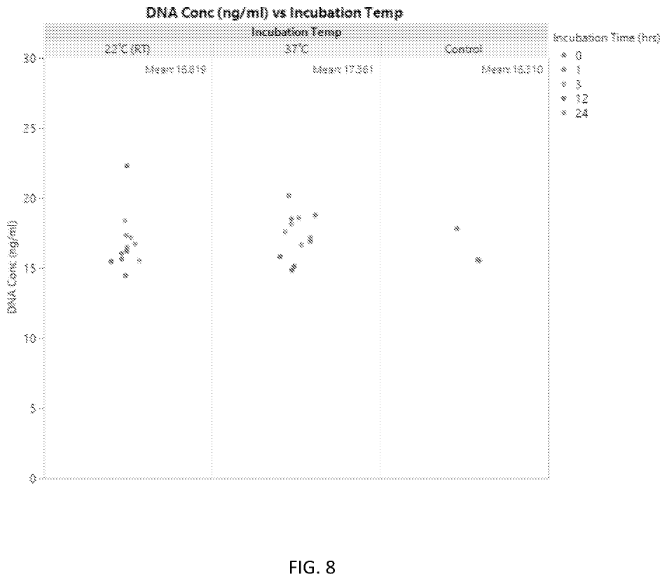

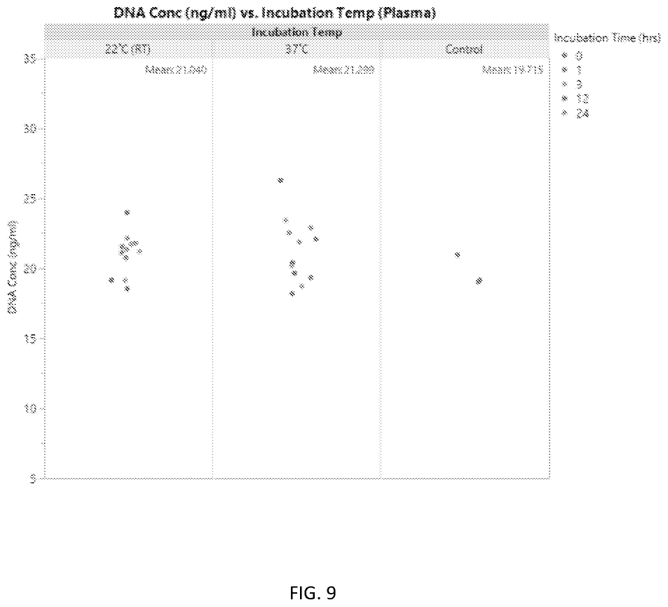

Procedure and Results: Two ml volumes of STEEN solution and plasma were spiked with sheared gDNA and incubated at both room temperature and 37° C. for 0, 1, 3, 12, and 24 hours. DNA was then extracted using an automated DNA extraction workflow. Eluates from the extractions were then measured using a reference gene qPCR method. Results are shown in FIG. 8 and FIG. 9.

Conclusions: The data in the figures above demonstrates that DNA is stable in

STEEN solution and plasma from 0-24 hours at both 22° C. (RT) and 37° C. This suggests that STEEN solution is a suitable matrix for maintaining DNA stability. While there is variance in the DNA concentrations of these samples, this variance within the variation expected when sample DNA is extracted.

### Example 9

### Quantification of Endogenous Total cfDNA Concentration in Collections from a Clinical EVLP Perfusate Taken at 1,2 and 3 hrs of Perfusion

Procedure and Results, Experiment I: Clinical EVLP perfusates from tubes 1-1 (hour 1), 2-1 (hour 2), and 3-1 (hour 3) were thawed at room temperature. Once tubes were completely thawed, they underwent a single high-speed centrifugation at 15000×g to pellet any residual debris (which was absent/minimal). Two ml aliquots of the supernatant were then extracted using an automated and clinically validated cfDNA extraction process for each sample in duplicate. Total cfDNA of samples was measured directly using a reference gene (RNAseP) qPCR method. Results are shown in Table 2.

TABLE 2
Reference gene qPCR measuring total cfDNA concentration
in EVLP clinical perfusate samples collected at hours
1, 2, 3, and a “0 hour” STEEN solution control.

| | Time on EVLP | Mean total cfDNA from |
|---|---|---|
| Matrix | Circuit (hr) | Perfusate (ng/ml) |
| STEEN Solution | 0 | 0.0 |
| Perfusate | 1 | 35.4 |
| Perfusate | 2 | 132.9 |
| Perfusate | 3 | 217.4 |

Conclusions: Perfusate samples drawn and processed have increasing levels of total cfDNA at hours 1, 2, and 3 of perfusion. Based the data demonstrating the stability of cfDNA in STEEN for up to 24 hours, this progressive increase in cfDNA concentration may result from progressive accumulation of cfDNA in the EVLP circuit over time.

### Example 10

### Quantification of Spiked-in Short Fragment DNA Control in Clinical EVLP Perfusate

Procedure and Results: Pre-centrifuged perfusates received in tubes 1-1 (hour 1), 2-1 (hour 2), and 3-1 (hour 3) were thawed at room temperature and then subjected to a single high-speed centrifugation at 15000×g to pellet any residual debris (noted to be absent-to-minimal). Two ml aliquots of each sample in duplicate were then spiked with a short fragment DNA control and extracted using a clinically validated automated cfDNA extraction process. The percentage recovery of short DNA fragment control was measured directly by qPCR to assure expected and consistent recovery. Results are shown in Table 3.

TABLE 3
qPCR measuring the percent recovery of spiked short fragment
DNA control from clinical EVLP perfusates at hours 1,
2, 3, and a “0 hour” STEEN solution control.

| | Time on EVLP | Percent Recovery Spiked |
|---|---|---|
| Matrix | Circuit (hr) | Short Fragment DNA Control |
| STEEN Solution | 0 | 57% |
| Perfusate | 1 | 58% |
| Perfusate | 2 | 57% |
| Perfusate | 3 | 54% |

Conclusions: The data in the table above demonstrates that short DNA fragments are extracted uniformly in clinical EVLP perfusate samples collected at 0, 1, 2, and 3 hrs at historically expected percentages. This data supports the conclusion that short fragment cfDNA is consistently extracted at 1-3 hrs of perfusion in STEEN solution, despite potentially varying matrix compositions, allowing confident comparison of total cfDNA levels at each of these timepoints.

### Example 11

### Evaluation of the Endogenous DNA Fragment Distribution in Clinical EVLP Perfusate Time Points Using a Method for Differentially Detecting Long and Short Fragments of DNA

Procedure and Results: Perfusates from tubes 1-1 (hour 1), 2-1 (hour 2), and 3-1 (hour 3) were thawed at room temperature. Once tubes were completely thawed, they underwent a single high-speed centrifugation at 15000×g. Two ml aliquots were extracted and analyzed using the short and long fragment DNA tests in triplicate. The extraction eluates were analyzed using a method for differentially detecting long and short fragments of DNA as a measure of the differential contributions of cellular apoptosis (the typical mode of cellular death in vivo that produces very short DNA fragments) versus cellular lysis (which produces longer DNA fragments). The proportion of long fragment DNA in the eluates (mean of duplicate determinations) is shown in Table 4 below.

TABLE 4
Long fragment DNA proportion of clinical EVLP
perfusates collected at hours 1, 2, and 3.

| | Time on EVLP | DNA Perfusate Long |
|---|---|---|
| Matrix | Circuit (hr) | Fragment Ratio Mean |
| Perfusate | 1 | 0.89 |
| Perfusate | 2 | 0.88 |
| Perfusate | 3 | 0.84 |

Conclusions: Extracted DNA in all examined perfusates contains a high proportion of long fragment DNA that exceeds the level which has been previously determined to indicate a significant degree of cellular (typically leukocyte) lysis. Note that the upper theoretical limit of this “long fragment ratio” ratio is 1.0 (at which point all DNA present would be much longer than that produced by apoptosis). As a reference, this ratio in heart transplant patients is less than 0.4 (usually around 0.2), assuming contaminating leukocytes have been promptly removed. Given that the centrifugation protocol was properly performed for these EVLP samples, we conclude that EVLP samples contain a preponderance of long fragment cfDNA, presumably as the result of cellular lysis rather than the apoptosis.

### Example 12

### Selective Quantification of Short Fragment cfDNA in a Clinical EVLP Perfusate After Selective Removal of Long Fragment DNA by Size-Selective, Bead-Based Purification (an Independent Measure of Short Fragment Accumulation Over Time During EVLP)

Procedure and Results: Clinical EVLP perfusates from tubes 1-2 (hour 1), 2-2 (hour 2), and 3-2 (hour 3) were thawed at room temperature. Once tubes were completely thawed, they underwent a single high-speed centrifugation at 15000×g. 100 μl perfusate aliquots were then purified using Promega ProNex® size selective purification system (NG2001 or NG2002) to preferentially isolate long fragment DNA. The remaining supernatant was then re-purified to preferentially isolate the remaining short fragment DNA.

Purified long and short fragment DNA eluates were then quantified using a reference gene qPCR method (Table 5). Additionally, the proportion of long fragment DNA in the eluates (mean of duplicate determinations) is shown in Table 6.

TABLE 5
Reference gene qPCR measuring DNA in EVLP clinical
perfusate samples after size-selected purification.

| | Time on | | Mean total cfDNA |
|---|---|---|---|
| | EVLP | | from Perfusate |
| Matrix | Circuit (hr) | Purification # | (ng/ml) |
| Perfusate | 1 | 1 (long fragment) | 51.43 |
| Perfusate | 2 | 1 (long fragment) | 120.18 |
| Perfusate | 3 | 1 (long fragment) | 212.85 |
| Perfusate | 1 | 2 (short fragment) | 1.59 |
| Perfusate | 2 | 2 (short fragment) | 3.29 |
| Perfusate | 3 | 2 (short fragment) | 9.82 |

TABLE 6
Long fragment DNA proportion of EVLP clinical
samples after size-selected bead purification.

| | Time on | | DNA Perfusate |
|---|---|---|---|
| | EVLP | | Long Fragment |
| Matrix | Circuit (hr) | Purification # | Ratio Mean |
| Perfusate | 1 | 1 (long fragment) | 0.90 |
| Perfusate | 2 | 1 (long fragment) | 0.88 |
| Perfusate | 3 | 1 (long fragment) | 0.87 |
| Perfusate | 1 | 2 (short fragment) | 0.56 |
| Perfusate | 2 | 2 (short fragment) | 0.58 |
| Perfusate | 3 | 2 (short fragment) | 0.52 |

Conclusions: Perfusates drawn and processed have increasing levels of both short and long fragment DNA at hours 1, 2, and 3, consistent with the progressive accumulation of cfDNA in the circuit predicted by the results reported in which the long-term stability of cfDNA during perfusion in STEEN was demonstrated (in contrast to the short half-life of cfDNA fragments in vivo due to active clearance by the liver). The dramatically higher levels of long fragment cfDNA in the EVLP samples detected by bead selection is consistent with the results of Example 11. Although comparatively low, there are also measurable levels of short fragment DNA in these EVLP perfusate samples which can be precisely compared between different EVLP procedures.

Elevated long-fragment cfDNA ratios appear to be a characteristic of EVLP perfusates, unlike in vivo blood collections in which apoptotic fragments dominate. Regardless, the overall total concentration of cfDNA in clinical EVLP circuits, coupled with the rate of accumulative increase in that level over time, is a valuable indicator of lung health during EVLP. In addition, using DNA fragment size selective analyses demonstrated to be feasible: correlation of short vs long cfDNA fragment lengths with EVLP lung health is shown to be practical in EVLP circuit collections.

It is understood that the foregoing detailed description and accompanying examples are merely illustrative and are not to be taken as limitations upon the scope of the disclosure.

Without limiting the disclosure, the following are example embodiments that may be used, formulated or applied:

Embodiment 1: A method of assessing the suitability of a potential graft for transplantation, comprising:
a) obtaining an amount of total short fragment cf-DNA and/or graft-specific short-fragment cf-DNA released from a graft prior to contacting the graft with blood cells from a potential recipient, and
b) assessing the amount(s) to determine the suitability of the graft for transplantation.

Embodiment 2: The method of Embodiment 1, wherein obtaining the amount comprises performing a method to quantify the amount of total short fragment cf-DNA and/or graft-specific short fragment cf-DNA released by the graft.

Embodiment 3: The method of Embodiment 1 or 2, wherein the method further comprises performing a method to quantify the amount of total short fragment cf-DNA and/or graft-specific short-fragment cf-DNA released by the graft subsequent to contacting the graft or cells thereof with blood cells from a potential recipient.

Embodiment 4: A method of assessing the suitability of a graft for transplantation, comprising: (a) obtaining an amount of total short fragment cf-DNA and/or graft-specific short fragment cf-DNA released by the graft subsequent to contacting of the graft or cells thereof with blood cells from a potential recipient, and (b) assessing the amount(s) to determine the suitability of the potential graft for transplantation.

Embodiment 5: The method of Embodiment 4, wherein obtaining the amount comprises performing a method to quantify the amount(s) of total short fragment cell-free DNA and/or graft-specific short fragment cf-DNA in order to obtain the amount.

Embodiment 6: The method of any one of Embodiments 1-5, wherein the method further comprises determining an amount of cell lysis.

Embodiment 7: The method of Embodiment 6, wherein the determining the amount of cell lysis comprises performing fragment analysis.

Embodiment 8: The method of Embodiment 7, wherein the fragment analysis comprising quantifying long and/or short fragments.

Embodiment 9: The method of any one of the preceding Embodiments, wherein the method further comprises one, two, three or more centrifugation steps of the sample(s) prior to obtaining the amount of total short fragment cf-DNA and/or graft-specific short-fragment cf-DNA and/or cell lysis.

Embodiment 10: The method of any one of the preceding Embodiments, wherein the method further comprises obtaining the potential graft or cells thereof.

Embodiment 11: The method of any one of the preceding Embodiments, wherein the method further comprises obtaining an amount of total short fragment cf-DNA and/or graft-specific short-fragment cf-DNA and/or determining the amount of cell lysis at one or more additional time points.

Embodiment 12: The method of any one of the preceding Embodiments, wherein the method further comprises comparing the amount(s) of the total short fragment cf-DNA and/or graft-specific cf-DNA and/or amount of cell lysis with threshold value(s) or amount(s) obtained from additional time point(s).

Embodiment 13: The method of any one of the preceding Emsbodiment, wherein the amount(s) of the total short fragment cf-DNA and/or graft-specific cf-DNA and/or amount of cell lysis are obtained in a sample of media in which the potential graft or cells therefrom are contained or are in contact with.

Embodiment 14: The method of Embodiment 13, wherein the media is a storage, perfusion, or preservation media in which the graft is contained or in contact with.

Embodiment 15: The method of Embodiment 13 or 14, wherein the cf-DNA is released from the graft into the media.

Embodiment 16: The method of any one of the preceding Embodiments, wherein the amount(s) is/are obtained in a perfusate sample.

Embodiment 17: The method of any one of the preceding Embodiments, wherein the amount(s) is/are obtained in an EVLP sample.

Embodiment 18: The method of any one of the preceding Embodiments, wherein the method further comprises obtaining the blood cells (e.g., blood) from the potential recipient.

Embodiment 19: The method of any one of the preceding Embodiments wherein the potential graft or cells thereof are from a potential donor of the same species as the potential recipient.

Embodiment 20: The method of any one of the preceding Embodiments, wherein the potential graft is an organ or organs.

Embodiment 21: The method of Embodiment 20, wherein the organ or organs comprise a heart, a lung, or a heart and a lung.

Embodiment 22: The method any one of the preceding Embodiments, wherein the method further comprises providing the values for the amount(s) of total cell-free DNA and/or graft-specific cf-DNA and/or cell lysis in a report.

Embodiment 23: The method of any one of the preceding Embodiments, wherein the method further comprises providing a determination about the suitability of the graft.

Embodiment 24: The method of any one of the preceding Embodiments, wherein the graft is monitored over time.

Embodiment 25: The method of Embodiment 24, wherein the graft is assessed every 15 minutes, every 30 minutes, hourly, daily, weekly, bimonthly or monthly prior to transplantation.

Embodiment 26: The method of any one of the preceding Embodiments, wherein the total short fragment cell-free DNA and/or graft-specific short fragment cf-DNA and/or cell lysis is measured using a quantitative amplification method.

Embodiment 27: The method of Embodiment 26, wherein the quantitation amplification method is selected from real-time PCR, digital PCR, and mismatch PCR.

Embodiment 28: The method of any one of Embodiments 1-25, wherein the amount of total short fragment cell-free DNA and/or graft-specific short fragment cf-DNA and/or cell lysis is measured using next-generation sequencing.

Embodiment 29: The method of any one of the preceding Embodiments, wherein when the value for the amount(s) are above a threshold value or a value from a prior point in time, decreasing suitability of the graft or unsuitability of the graft for transplantation is indicated.

Embodiment 30: The method of any one of the preceding Embodiments, wherein when the value for the amount(s) are below a threshold value or a value from a prior point in time, increasing suitability of the graft or suitability of the graft for transplantation is indicated.

Embodiment 31: The method of any one of the preceding Embodiments, further comprising performing treatment of the graft, potential donor, and/or recipient, or providing information regarding such a treatment.

Embodiment 32: A report comprising the value(s) or amount(s) of total short fragment cell-free DNA, specific short fragment cell-free DNA, and/or cell lysis obtained by the method of any one of the preceding Embodiments.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

1. A method of assessing the suitability of a potential graft for transplantation, comprising:
a. obtaining an amount of total short fragment cf-DNA and/or graft-specific short-fragment cf-DNA released from a graft prior to contacting the graft with blood cells from a potential recipient, and
b. assessing the amount(s) to determine the suitability of the graft for transplantation.

2. The method of claim 1, wherein obtaining the amount comprises performing a method to quantify the amount of total short fragment cf-DNA and/or graft-specific short fragment cf-DNA released by the graft.

3. The method of claim 1 wherein the method further comprises performing a method to quantify the amount of total short fragment cf-DNA and/or graft-specific short-fragment cf-DNA released by the graft subsequent to contacting the graft or cells thereof with blood cells from a potential recipient.

4. A method of assessing the suitability of a graft for transplantation, comprising:
a. obtaining an amount of total short fragment cf-DNA and/or graft-specific short fragment cf-DNA released by the graft subsequent to contacting of the graft or cells thereof with blood cells from a potential recipient, and
b. assessing the amount(s) to determine the suitability of the potential graft for transplantation.

5. The method of claim 4, wherein obtaining the amount comprises performing a method to quantify the amount(s) of total short fragment cell-free DNA and/or graft-specific short fragment cf-DNA in order to obtain the amount.

6. The method of claim 1, wherein the method further comprises determining an amount of cell lysis.

7. The method of claim 6, wherein the determining the amount of cell lysis comprises performing fragment analysis, wherein the fragment analysis comprising quantifying long and/or short fragments.

8. (canceled)

9. The method of claim 1, wherein the method further comprises one, two, three or more centrifugation steps of the sample(s) prior to obtaining the amount of total short fragment cf-DNA and/or graft-specific short-fragment cf-DNA and/or cell lysis.

10. (canceled)

11. The method of claim 1, wherein the method further comprises obtaining an amount of total short fragment cf-DNA and/or graft-specific short-fragment cf-DNA and/or determining the amount of cell lysis at one or more additional time points.

12. The method of claim 1, wherein the method further comprises comparing the amount(s) of the total short fragment cf-DNA and/or graft-specific cf-DNA and/or amount of cell lysis with threshold value(s) or amount(s) obtained from additional time point(s).

13. The method of claim 1, wherein the amount(s) of the total short fragment cf-DNA and/or graft-specific cf-DNA and/or amount of cell lysis are obtained in a sample of media in which the potential graft or cells therefrom are contained or are in contact with.

14. The method of claim 13, wherein the media is a storage, perfusion, or preservation media in which the graft is contained or in contact with, wherein the cf-DNA is released from the graft into the media.

15. (canceled)

16. The method of claim 1, wherein the amount(s) is/are obtained in a perfusate sample or an EVLP sample.

17.-18. (canceled)

19. The method of claim 1, wherein the potential graft or cells thereof are from a potential donor of the same species as the potential recipient.

20. The method of claim 1, wherein the potential graft is an organ or organs comprising a heart, a lung, or a heart and a lung.

21-23. (canceled)

24. The method of claim 1, wherein the graft is monitored every 15 minutes, every 30 minutes, hourly, daily, weekly, bimonthly or monthly prior to transplantation.

25. (canceled)

26. The method of claim 1, wherein the total short fragment cell-free DNA and/or graft-specific short fragment cf-DNA and/or cell lysis is measured using a quantitative amplification method.

27. (canceled)

28. The method of claim 1, wherein the amount of total short fragment cell-free DNA and/or graft-specific short fragment cf-DNA and/or cell lysis is measured using next-generation sequencing.

29. The method of claim 1, wherein when the value for the amount(s) are above a threshold value or a value from a prior point in time, decreasing suitability of the graft or unsuitability of the graft for transplantation is indicated; or wherein when the value for the amount(s) are below a threshold value or a value from a prior point in time, increasing suitability of the graft or suitability of the graft.

30. (canceled)

31. The method of claim 1, further comprising performing treatment of the graft, potential donor, and/or recipient.

32-33. (canceled)
